(19) United States
(12) Patent Application Publication
(10) Pub. No.: US 2023/0090556 A1
(43) Pub. Date: Mar. 23, 2023

(54) METHODS, MEDIUMS, AND SYSTEMS FOR ESTABLISHING A QUALITY CONTROL RECORD CHAIN FOR LABORATORY ANALYTICAL INSTRUMENTS

(71) Applicant: Waters Technologies Ireland Limited, Dublin 2 (IE)

(72) Inventor: Matthew Wherry, High Peak (GB)

(73) Assignee: Waters Technologies Ireland Limited, Dublin 2 (IE)

(21) Appl. No.: 17/932,845

(22) Filed: Sep. 16, 2022

Related U.S. Application Data
(60) Provisional application No. 63/245,515, filed on Sep. 17, 2021.

(51) Int. Cl. H04L 9/00; G06F 21/60; H04L 9/32; G16H 10/40; G01N 35/00

(52) U.S. Cl. CPC H04L 9/50; G06F 21/602; H04L 9/321; G16H 10/40; G01N 35/00623

(57) ABSTRACT

Exemplary embodiments provide a chain of authority linking records of auditable parameters for analytical laboratory instruments. The linked records provide a complete picture of the various items that contributed to a set of experimental results, allowing a user to (for example) trace a problem in an experiment back to its source, even if the source lies with a third party. This increases confidence in the quality of analytical results, simplifies audit and reporting compliance, and improves the traceability of instruments, reagents, and services.

### CROSS-REFERENCE TO RELATED APPLICATIONS

This application claims the benefit of and priority to U.S. Provisional Patent Application No. 63/245,515, filed on Sep. 17, 2021, the entire contents of which are incorporated by reference.

### BACKGROUND

Laboratory analytical instruments are devices for qualitatively and/or quantitatively analyzing samples and are often used in a laboratory setting for scientific research or testing. Such devices may measure the chemical makeup of a sample, the quantity of components in a sample, and perform similar analyses of interest. Examples include mass spectrometers, chromatographs, titrators, spectrometers, elemental analyzers, particle size analyzers, rheometers, thermal analyzers, etc.

Many factors go into an experimental analysis using a laboratory analytical instrument. For example, the quality of construction of the laboratory analytical instrument, the accuracy of its calibration, the quality of the samples analyzed, the particular analysis method used, and many other parameters may affect the accuracy of the analysis.

It is often desirable, when evaluating a particular experimental result, to be able to review these parameters to ensure that they fall within accepted ranges. However, they are not all within the direct control of the laboratory performing the analysis. For example, the sample being tested may have been produced by a third party, using reagents produced by a different third party. The reagents may have been tested for quality by a laboratory analytical instrument different from the one performing the current experiment and located in a different lab. The quality control records for the sample may thus be distributed among several different entities, and the laboratory conducting the current analysis may not have access to all (or any) of them. Similarly, a device manufacturer may hold the records for the production and maintenance of the laboratory analytical instrument, and the lab may use an analytical method created and maintained by a scientific organization. Identifying and retrieving these records can be a cumbersome and difficult process, if they can be obtained at all.

### BRIEF SUMMARY

Exemplary embodiments relate to computer-implemented methods, as well as non-transitory computer-readable mediums storing instructions for performing the methods, apparatuses configured to perform the methods, etc.

In one aspect, a computer implemented method includes receiving analytical data from a laboratory analytical instrument, where the analytical data is generated based on auditable parameter. An auditable parameter may be any parameter known to be capable of affecting the quality or accuracy of the analytical data and for which records can be generated for review after the data is analyzed. An auditable parameter may be a parameter that is required to be monitored or recorded for purposes of complying with auditing requirements of a regulatory authority, scientific body, corporation, etc. An auditable parameter may be a specific value, such as a setting, but may also be a more general identifier (such as an identification of the specific instrument used to generate the data or an identification of a sample or standard being analyzed).

A record for the auditable parameter may be accessed, where the record includes a chain of authority tracing the auditable parameter backwards to a previous auditable parameter. The chain of authority may be represented as a tree or linked list. In the chain of authority, the record of the current auditable parameter may refer back to the previous auditable parameter in a verifiable way, so that the record of the previous auditable parameter cannot be modified without the modification being detected. More than one previous auditable parameter may be associated with any given auditable parameter, and previous auditable parameters may also have their own predecessor auditable parameters.

The record may be cryptographically secured based on the chain of authority to generate a cryptographically secured record, and the cryptographically secured record may be associated with the data. In one example, the record may be cryptographically secured by calculating a cryptographic hash over the data and/or previous data or records in the chain of authority. The data in the record may be combined with the hash and stored with the data. In some embodiments, the cryptographically secured record may be embedded with the data so that the two are part of the same data structure; retrieving the data may cause the cryptographically secured record to be retrieved at the same time.

The auditable parameter may include an identification of at least one of a reagent, an instrument, a method, a calibration, or a quality control action. A reagent may be a sample being tested by the laboratory analytical instrument or may be a constituent of the sample. An instrument may be a laboratory analytical instrument. The method may be an analytical method or workflow that describes how the sample is measured or how the resulting data is analyzed. A calibration may refer to a technique for calibrating the laboratory analytical instrument, or a record generated from a particular calibration. A quality control action may be an action to test or adjust the laboratory analytical instrument using a known standard (a known chemical compound used to test the performance of the instrument at regular intervals).

The record may be compliant with an audit requirement defined by a regulatory or scientific authority. The regulatory or scientific authority may define when records must be kept or certain actions must be recorded (for example, moving backwards in an analytical workflow to re-analyze experimental data with new settings). The record may satisfy the requirements of the authority for purposes of creating an audit trail.

The record may trace back to a root that includes a standard, a pharmacopeia, a scientific paper, a regulation, or design data for a reagent or a part of the laboratory analytical instrument. When the chain of authority is followed all the way back to such a root, a complete overview of the factors going into a certain auditable parameter can be traced.

The auditable parameter and previous auditable parameter may each be associated with a quality gate that triggers a respective record to be created. A quality gate may be represented by transitions in an analytical workflow (where responsibility for the data being analyzed passes from one user to a different user). Crossing the transition may trigger the creation of a new quality control record. Other types of quality gates may also be used. For example, whenever internal practices require that a document or result be signed, the signature requirement may represent a quality gate that triggers the creation of a new quality control record. This may be used in situations where an engineer needs to sign off on a design, a quality control specialist signs off on a test of a reagent or sample, when an item is shipped to a customer, when maintenance is carried out, etc. Not all quality gates require the presence of a signature; they may be triggered, for instance, by the creation of an auditable document such as an invoice, shipping record, an academic paper, a standard from a scientific organization, etc.

The record may be in the form of a tree having nodes secured by a cryptographic hash based on respective content of one or more preceding nodes. For instance, the record may take the form of a Merkle tree or blockchain. The record may take the form of a distributed ledger.

At least one record along the chain of authority may be maintained by a third party. The method may further include receiving a request to access the at least one record. The request may be transmitted to the third party. The at least one record may be received in response to the request, where the at least one record is secured by a cryptographic hash maintained by or associated with the third party responsible for maintaining the record. The at least one record may be validated using the cryptographic hash. In this way, the auditable parameter can be traced back to records that may not be in the possession of the laboratory analyzing the sample (and those records can be traced back to further third parties). Thus, a laboratory can quickly and efficiently identify and retrieve the records that describe how a particular analytical result was generated.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an example of a mass spectrometry system according to an exemplary embodiment.

FIG. 2 illustrates an example of a workflow that operates on acquired data in accordance with one embodiment.

FIG. 3 illustrates an example of parameters that may affect the accuracy or quality of an experimental result in accordance with one embodiment.

FIG. 4 illustrates an example of a record chain for parameters affecting the production and maintenance of a laboratory analytical instrument in accordance with one embodiment.

FIG. 5 illustrates an example of a chain of parameters affecting an analytical method in accordance with one embodiment.

FIG. 6A illustrates an example of a chain of parameters affecting a sample in accordance with one embodiment.

FIG. 6B illustrates an example of a chain of parameters affecting a sample in accordance with one embodiment.

FIG. 7A is a flowchart depicting exemplary logic for creating a quality control record in accordance with one embodiment.

FIG. 7B is a flowchart depicting exemplary logic for retrieving quality control records in accordance with one embodiment.

FIG. 8 depicts an illustrative computer system architecture that may be used to practice exemplary embodiments described herein.

### DETAILED DESCRIPTION

Exemplary embodiments provide a chain of authority linking records of auditable parameters. The linked records provide a complete picture of the various items that contributed to a set of experimental results, allowing a user to (for example) trace a problem in an experiment back to its source, even if the source lies with a third party. This increases confidence in the quality of analytical results, simplifies audit and reporting compliance, and improves the traceability of instruments, reagents, and services.

As an aid to understanding, a series of examples will first be presented before detailed descriptions of the underlying implementations are described. It is noted that these examples are intended to be illustrative only and that the present invention is not limited to the embodiments shown.

Reference is now made to the drawings, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form in order to facilitate a description thereof. The intention is to cover all modifications, equivalents, and alternatives consistent with the claimed subject matter.

In the Figures and the accompanying description, the designations “a” and “b” and “c” (and similar designators) are intended to be variables representing any positive integer. Thus, for example, if an implementation sets a value for a=5, then a complete set of components 122 illustrated as components 122-1 through 122-a may include components 122-1, 122-2, 122-3, 122-4, and 122-5. The embodiments are not limited in this context.

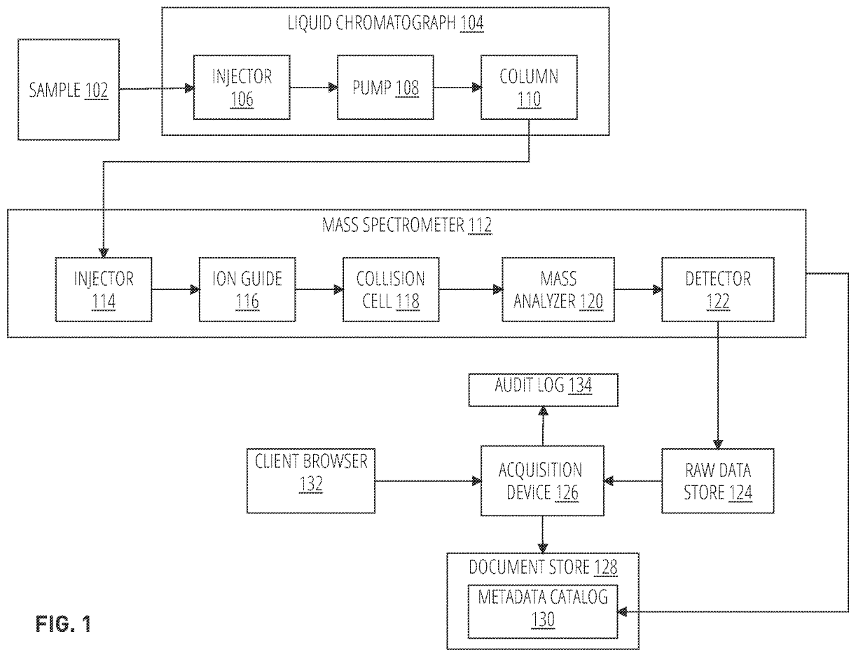

For purposes of illustration, FIG. 1 is a schematic diagram of a system that may be used in connection with techniques herein. Although FIG. 1 depicts particular types of devices in a specific LCMS configuration, one of ordinary skill in the art will understand that different types of chromatographic devices (e.g., LC, MS, tandem MS, etc.) may also be used in connection with the present disclosure. Exemplary embodiments may also be used in conjunction with other data sources than the ones depicted and described in detail herein, such as large scale chromatography (such as the GE Akta system), NMR, IR, CE etc.

A sample 102 is injected into a liquid chromatograph 104 through an injector 106. A pump 108 pumps the sample 102 through a column 110 to separate the mixture into component parts according to retention time through the column.

The output from the column is input to a mass spectrometer 112 for analysis. Initially, the sample is desolved and ionized by a desolvation/ionization device 114. Desolvation can be any technique for desolvation, including, for example, a heater, a gas, a heater in combination with a gas or other desolvation technique. Ionization can be by any ionization techniques, including for example, electrospray ionization (ESI), atmospheric pressure chemical ionization (APCI), matrix assisted laser desorption (MALDI) or other ionization technique. Ions resulting from the ionization are fed to a collision cell 118 by a voltage gradient being applied to an ion guide 116. Collision cell 118 can be used to pass the ions (low-energy) or to fragment the ions (high-energy).

Different techniques (including one described in U.S. Pat. No. 6,717,130, to Bateman et al., which is incorporated by reference herein) may be used in which an alternating voltage can be applied across the collision cell 118 to cause fragmentation. Spectra are collected for the precursors at low-energy (no collisions) and fragments at high-energy (results of collisions).

The output of collision cell 118 is input to a mass analyzer 120. Mass analyzer 120 can be any mass analyzer, including quadrupole, time-of-flight (TOF), ion trap, magnetic sector mass analyzers as well as combinations thereof. A detector 122 detects ions emanating from mass analyzer 120. Detector 122 can be integral with mass analyzer 120. For example, in the case of a TOF mass analyzer, detector 122 can be a microchannel plate detector that counts intensity of ions, i.e., counts numbers of ions impinging it.

A raw data store 124 may provide permanent storage for storing the ion counts for analysis. For example, raw data store 124 can be an internal or external computer data storage device such as a disk, flash-based storage, and the like. An acquisition device 126 analyzes the stored data. Data can also be analyzed in real time without requiring storage in a storage medium 124. In real time analysis, detector 122 passes data to be analyzed directly to computer 126 without first storing it to permanent storage.

Collision cell 118 performs fragmentation of the precursor ions. Fragmentation can be used to determine the primary sequence of a peptide and subsequently lead to the identity of the originating protein. Collision cell 118 includes a gas such as helium, argon, nitrogen, air, or methane. When a charged precursor interacts with gas atoms, the resulting collisions can fragment the precursor by breaking it up into resulting fragment ions.

Metadata describing various parameters related to data acquisition may be generated alongside the raw data. This information may include a configuration or identification of the liquid chromatograph 104 or mass spectrometer 112 (or other chromatography apparatus that acquires the data), which may define a data type, temperatures (e.g., of the laboratory or LC system), location (e.g., of the laboratory or LC system within a laboratory), batch or lot codes associated with the materials used in generation of the data acquisition, and others discussed in more detail below. An identifier (e.g., a key) for a codec that is configured to decode the data may also be stored as part of the metadata and/or with the raw data. The metadata may be stored in a metadata catalog 130 in a document store 128.

The acquisition device 126 may operate according to a workflow, providing visualizations of data to an analyst at each of the workflow steps and allowing the analyst to generate output data by performing processing specific to the workflow step. The workflow may be generated and retrieved via a client browser 132. As the acquisition device 126 performs the steps of the workflow, it may read raw data from a stream of data located in the raw data store 124. As the acquisition device 126 performs the steps of the workflow, it may generate processed data that is stored in a metadata catalog 130 in a document store 128; alternatively or in addition, the processed data may be stored in a different location specified by a user of the acquisition device 126. It may also generate audit records that may be stored in an audit log 134.

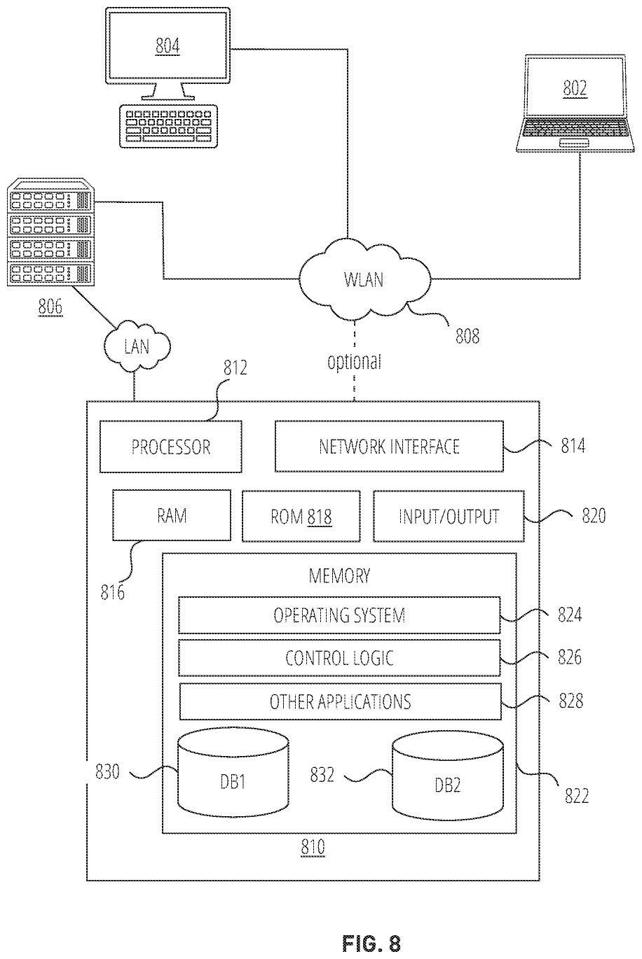

The exemplary embodiments described herein may be performed at the client browser 132 and acquisition device 126, among other locations. An example of a device suitable for use as an acquisition device 126 and/or client browser 132, as well as various data storage devices, is depicted in FIG. 8. Servers and other computer hardware can either be on a local network or using cloud technology.

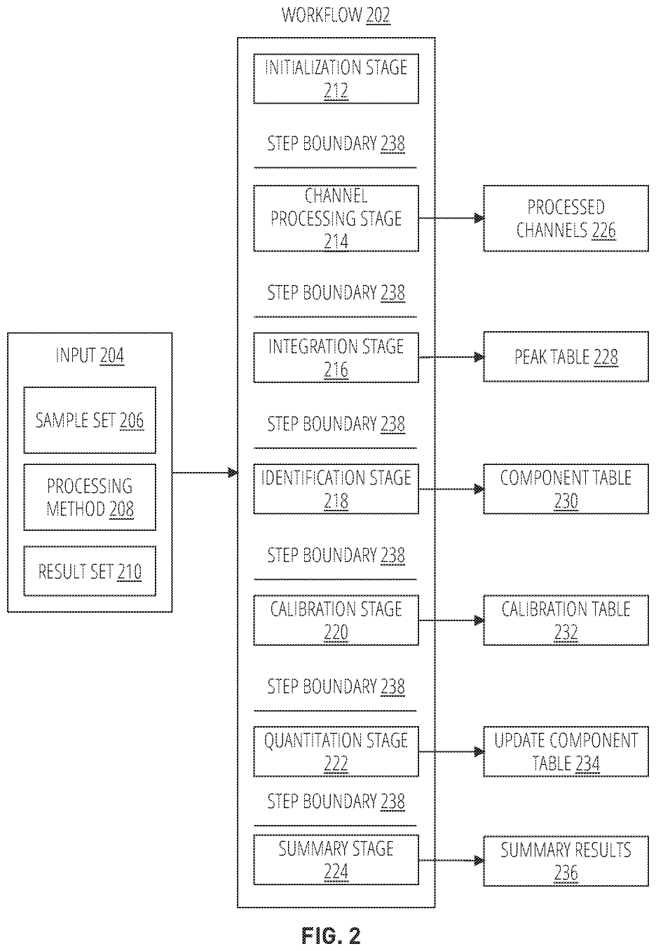

For context, FIG. 2 depicts a simplified example of a workflow 202 that may be applied by the acquisition device 126 of FIG. 1. The workflow 202 is designed to take a set of inputs 204, apply a number of workflow steps or stages to the inputs to generate outputs at each stage, and continue to process the outputs at subsequent stages in order to generate results of the experiment. It is noted that the workflow 202 is a specific example of a workflow, and includes particular stages performed in a particular order. However, the present invention is not limited to the specific workflow depicted in FIG. 2. Other suitable workflows may have more, fewer, or different stages performed in different orders.

The initial set of inputs 204 may include a sample set 206, which includes the raw (unprocessed) data received from the chromatography experimental apparatus. This may include measurements or readings, such as mass-to-charge ratios. The measurements that are initially present in the sample set 206 may be measurements that have not been processed, for example to perform peak detection or other analysis techniques. The sample set 206 may include data in the form of a stream (e.g., a sequential list of data values received in a steady, continuous flow from an experimental apparatus).

In the context of the present application, the sample set 206 may represent the raw data stored in the raw data store 124 and returned by the endpoint interface. The sample set 206 may be represented as a model of a data stream (e.g., including data structures corresponding to data points gathered by the chromatography apparatus). The workflow 202 may be performed on the sample set 206 data by an application running on the acquisition device 126 and/or running within a data ecosystem.

The initial set of inputs 204 may also include a processing method 208, which may be a template method (as discussed above) that is applied to (and hence embedded in) the workflow 202. The processing method 208 may include settings to be applied at various stages of the workflow 202.

The initial set of inputs 204 may also include a result set 210. When created, the result set 210 may include the information from the sample set 206. In some cases, the sample set 206 may be processed in some initial manner when copied into the result set 210—for example, MS data may require extracting, smoothing, etc. before being provided to a workflow 202. The processing applied to the initial result set 210 may be determined on a case-by-case basis based on the workflow 202 being used. Once the raw data is copied from a sample set 206 to create a result set 210, that result set 210 may be entirely independent from the sample set 206 for the remainder of its lifecycle.

The workflow 202 may be divided into a set of stages. Each stage may be associated with one or more stage processors that perform calculations related to that stage. Each stage processor may be associated with stage settings that affect how the processor generates output from a given input.

Stages may be separated from each other by step boundaries 238. The step boundaries 238 may represent points at which outputs have been generated by a stage and stored in the result set, at which point processing may proceed to the next stage. Some stage boundaries may require certain types of input in order to be crossed (for example, the data generated at a given stage might need to be reviewed by one or more reviewers, who need to provide their authorization in order to cross the step boundary 238 to the next stage). Step boundaries 238 may apply any time a user moves from one stage to a different stage, in any direction. For example, a step boundary 238 exists when a user moves from the initialization stage 212 to the channel processing stage 214, but also exists when a user attempts to move backwards from the quantitation stage 222 back to the integration stage 216. Step boundaries 238 may be ungated, meaning that once a user determines to move to the next stage no further input (or only a cursory input) is required, or gated, meaning that the user must provide some sort of confirmation indicating that they wish to proceed to a selected stage (perhaps in response to a warning raised by the acquisition device 126), or a reason for moving to a stage, or credentials authorizing the workflow 202 to proceed to the selected stage.

In an initialization stage 212, each of the stage processors may respond by clearing the results that it generates. For example, the stage processor for the channel processing stage 214 may clear all its derived channels and peak tables (see below). At any point in time, clearing a stage setting may clear stage tracking from the current stage and any subsequent stage. In this example, the initialization stage 212 does not generate any output.

After crossing a step boundary 238, processing may proceed to a channel processing stage 214. As noted above, chromatography detectors may be associated with one or more channels on which data may be collected. At the channel processing stage 214, the acquisition device 126 may derive a set of processing channels present in the data in the result set 210 and may output a list of processed channels 226. The list of processed channels 226 may be stored in a versioned sub-document associated with the channel processing stage 214, which may be included in the result set 210.

After crossing a step boundary 238, processing may proceed to an integration stage 216, which identifies peaks in the data in the result set 210 based on the list of processed channels 226. The integration stage 216 may identify the peaks using techniques specified in the settings for the integration stage 216, which may be defined in the processing method 208. The integration stage 216 may output a peak table 228 and store the peak table 228 in a versioned sub-document associated with the integration stage 216. The sub-document may be included in the result set 210.

After crossing a step boundary 238, processing may proceed to identification stage 218. In this stage, the acquisition device 126 may identify components in the mixture analyzed by the chromatography apparatus based on the information in the peak table 228. The identification stage 218 may output a component table 230, which includes a list of components present in the mixture. The component table 230 may be stored in a versioned sub-document associated with the identification stage 218. The sub-document may be included in the result set 210.

After crossing a step boundary 238, processing may proceed to calibration stage 220. During a chromatography experiment, calibration compounds may be injected into the chromatography apparatus. This process allows an analyst to account for subtle changes in electronics, cleanliness of surfaces, ambient conditions in the lab, etc. throughout an experiment. In the calibration stage 220, data obtained with respect to these calibration compounds is analyzed and used to generate a calibration table 232, which allows the acquisition device 126 to make corrections to the data to ensure that it is reliable and reproducible. The calibration table 232 may be stored in a versioned sub-document associated with the calibration stage 220. The sub-document may be included in the result set 210.

After crossing a step boundary 238, processing may proceed to quantitation stage 222. Quantitation refers to the process of determining a numerical value for the quantity of an analyte in a sample. The acquisition device 126 may use the results from the previous stages in order to quantify the components included in the component table 230. The quantitation stage 222 may update 234 the component table 230 stored in the result set 210 with the results of quantitation. The updated component table 230 may be stored in a versioned sub-document associated with the quantitation stage 222. The sub-document may be included in the result set 210.

After crossing a step boundary 238, processing may proceed to summary stage 224. In the summary stage 224, the results of each of the previous stages may be analyzed and incorporated into a report of summary results 236. The summary results 236 may be stored in a versioned sub-document associated with the summary stage 224. The sub-document maybe included in the result set 210.

As used herein, a step may correspond to the above-noted stages. Alternatively, a single stage may include multiple steps, or multiple stages may be organized into a single step. In any event, all the activities performed in a given step should be performable by the same user or group of users, and each step is associated with one or more pages that describe a set of configuration options for the step (e.g., visualization options, review options, step configuration settings, etc.)

There may be a transition at some or all of the step boundaries 238, although not every step boundary 238 need be a transition. A transition may signify a change in responsibility for a set of data from a first user or group of users to a second, distinct user or group of users.

As used herein, a quality gate may be represented by the transitions in the workflow 202, where crossing the transition may trigger the creation of a new quality control record. Other types of quality gates may also be used. For example, whenever internal practices require that a document or result be signed, the signature may represent a quality gate that triggers the creation of a new quality control record. This may be used in situations where an engineer needs to sign off on a design, a quality control specialist signs off on a test of a reagent or sample, when an item is shipped to a customer, when maintenance is carried out, etc. Not all quality gates require the presence of a signature; they may be triggered, for instance, by the creation of an auditable document such as an invoice, shipping record, an academic paper, a standard from a scientific organization, etc.

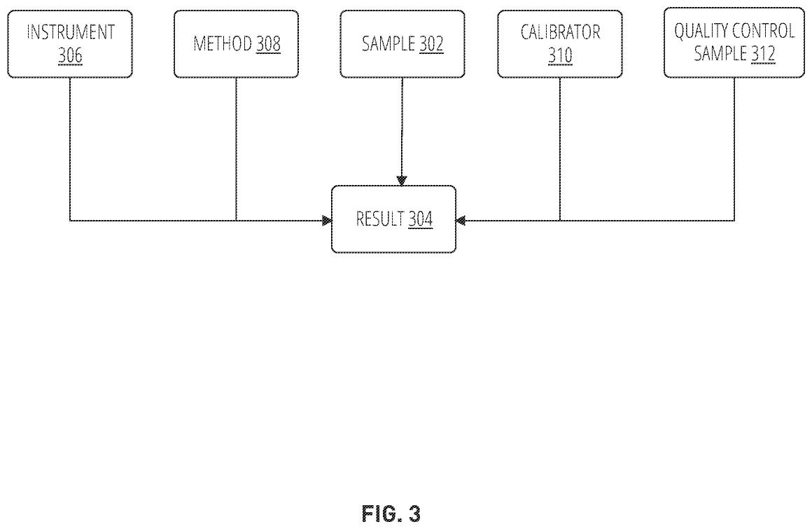

FIG. 3 depicts examples of various auditable parameters that affect the accuracy or quality of an analytical result 304. The result 304 may be, for instance, an analysis of a sample 302; when a record of this auditable parameter is generated, the record may include information used to identify the sample (e.g., an identification number or code, a batch or lot number, the type of chemical compound making up the sample, the manufacturer of the sample, etc.).

Factors such as the quality or purity of the sample 302, date or location of manufacture of the sample 302, identity of the manufacturer or supplier of sample 302, reference data regarding expected sample 302 characteristics, are some of the most readily apparent examples of parameters affecting the result 304, but other factors can also have a major impact on the result 304.

For example, the instrument 306 used to test the sample may have a defect or may not have been optimally maintained, which can decrease confidence in the result 304. A record for an instrument 306 auditable parameter may include an identifier of the specific instrument 306, available maintenance records, available calibration records, a manufacturer of the instrument 306, a type of the instrument 306, components or subassemblies of the instrument 306, identifying information regarding the revision or version number of the instrument 306 and/or its components or subassemblies, where or when the instrument 306 and/or its components or subassemblies were manufactured, etc.

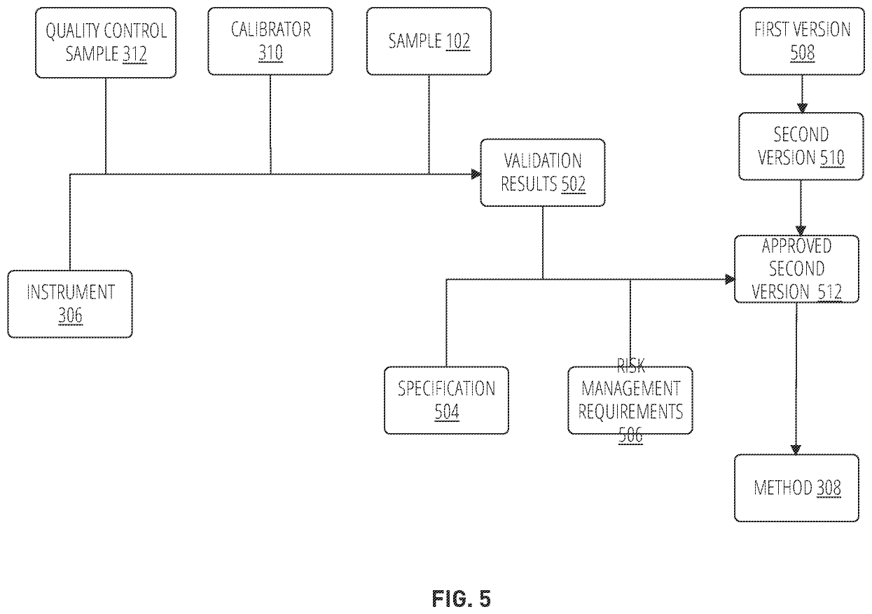

A method 308 may represent a sequence of steps performed in connection with the instrument 306, sample 302, calibrator 310, and/or quality control sample 312. A record for method 308 may include further information as described herein with regard to FIG. 5, such as the operator or operator(s) who performed certain steps of the method, the time or date that such steps were performed, and the version of the method that was performed.

A calibrator 310 may represent a chemical compound used to calibrate the instrument 306; the results of analyzing the calibrator 310 with the instrument 306 may be used to adjust settings of the instrument 306 or to adjust the output data. The calibrator 310 may be a known reference compound or a standard. A record for a calibrator 310 may include similar information to that of the sample 302.

Quality control samples 312 may be used for a variety of purposes. For instance, whereas a calibrator 310 is used to determine that the instrument 306 is performing as intended by the manufacturer across operating ranges defined by the lab, a quality control sample 312 may be used to determine that the analytical method 308 is suitable for its intended purpose at the time the sample 302 is tested. Quality control samples quality control sample 312 can thus be used to perform system suitability testing (SST). In another example, a quality control sample 312 can be used to perform a sample suitability test. In this case, the quality control sample 312 may be the same compound as the sample 302 or the calibrator 310 but may have a known concentration or relative potency. The quality control sample 312 should therefore generate results that are parallel to the sample 302 or calibrator 310; a failure to achieve parallel results may indicate that one of the compounds tested has degraded or that the instrument 306 is not functioning as intended. A record for a quality control sample 312 may include similar information to that of the sample 302.

The depiction of auditable parameters in FIG. 3 is relatively simple. However, in practice each of the auditable parameters may be tied to or may include a network or web of interrelated information maintained by many different parties.

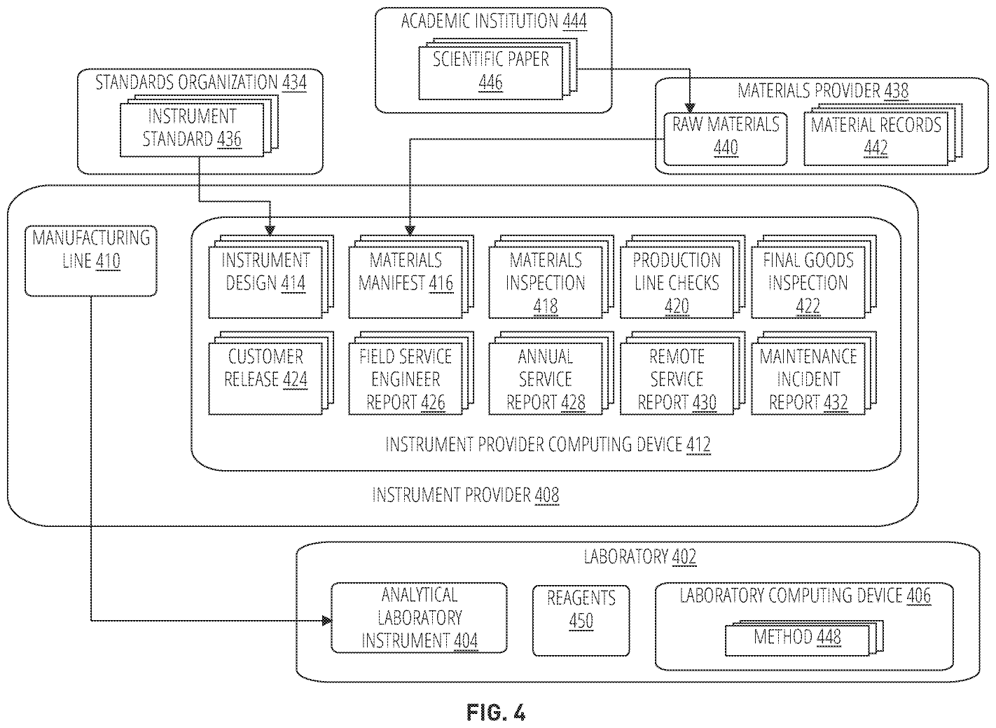

For instance, FIG. 4 shows some of the information and records that may be of interest to determine whether an analytical laboratory instrument 404 is producing accurate results.

The analytical laboratory instrument 404 may be located in a laboratory 402 and under the control of a laboratory administrator. The laboratory administrator may also be responsible for maintaining a laboratory computing device 406, which may store and analyze data from the analytical laboratory instrument 404. The laboratory computing device 406 may be collocated with the analytical laboratory instrument 404 within the laboratory 402 or may be located remote from the laboratory 402 (e.g., the laboratory computing device 406 may be a cloud computing device).

The analytical laboratory instrument 404 may have been manufactured or distributed by an instrument provider 408, which may be a different entity than the laboratory 402. The analytical laboratory instrument 404 may have been produced on a manufacturing line 410 at the instrument provider 408. Records relating to the production, delivery, and maintenance of the analytical laboratory instrument 404 may be stored in an instrument provider computing device 412 administered by an administrator of the instrument provider 408. The instrument provider computing device 412 may be collocated with the manufacturing line 410 at the instrument provider 408 or may be remote from the instrument provider 408.

The analytical laboratory instrument 404 may have been produced according to an instrument design 414. The instrument design 414 may, in turn, be based on one or more instrument standards 436 maintained by one or more standards organizations 434 (or could be an implementation of an idea in a scientific paper 446 maintained by an academic institution 444). A standards organization 434 may be an academic or technological entity that produces sets of rules or guidelines (a standard). In this example, the instrument standard 436 may be a set of rules or guidelines for how an analytical laboratory instrument 404 should operate to produce a measurement, tolerances for the production of various components of the analytical laboratory instrument 404, suitable materials to be used in the production, etc.

If there is a problem with the analytical laboratory instrument 404, the problem may lie in the original design of the analytical laboratory instrument 404, which would be reflected in the instrument design 414. It may be, for instance, that the instrument design 414 is not a proper reflection of the instrument standard 436. Alternatively, the instrument design 414 may properly implement instrument standard 436, but the problem may arise from a scenario not contemplated by the instrument standard 436. Thus, it may be helpful to be able to trace the design of the analytical laboratory instrument 404 back to the instrument standard 436. In this case, the chain of authority may include the instrument provider 408 and the standards organization 434, and it may be necessary to retrieve documents from these third parties in order to trace back the relevant records.

The instrument design 414 may serve as the design document for many instruments that were produced on the manufacturing line 410, of which the analytical laboratory instrument 404 may be one (e.g., other instruments implementing the instrument design 414 may have been produced and sold to other customers). The instrument design 414 may accordingly be associated with a record (e.g., a node in a Merkle tree or a record in a blockchain) that serves as a parent node from which multiple branches derive (where each branch represents a different instrument produced on the manufacturing line 410 according to the instrument design 414, but potentially using different materials, having different service records, etc.).

The analytical laboratory instrument 404 may have been produced on the manufacturing line 410 using a set of raw materials 440 provided by a materials provider 438. When delivered from the materials provider 438 to the instrument provider 408, the raw materials 440 may have been logged in a materials manifest 416, and then inspected by the instrument provider 408. The inspection results may be stored in a materials inspection 418. Prior to delivery, the raw materials 440 may also have been inspected by the materials provider 438, which may maintain its own material records 442. The formulation of the raw materials 440 may have been described in one or more scientific papers 446 produced by an academic institution 444 (or in a standard maintained by a standards organization 434).

Problems in the analytical laboratory instrument 404 may derive from the materials used to produce the analytical laboratory instrument 404, and the materials manifest 416, materials inspection 418, and material records 442 may each be stored as records in the chain of authority. By consulting these records, the problem can be traced back to its origin. For example, it is possible that the raw materials 440 were defective when originally produced, which would be reflected in the material records 442. Alternatively, they may have been corrupted in transit to the instrument provider 408 (or afterwards), which could be reflected in the materials manifest 416 or materials inspection 418. If a user wishes to determine whether the raw materials 440 used were in fact suitable for purposes of building an analytical laboratory instrument 404, the user could trace back the raw materials 440 to the scientific paper 446 that described their acceptable uses.

As the analytical laboratory instrument 404 is produced on the manufacturing line 410, there may be other documents generated. For example, before, during, or after the production of the 404 on the manufacturing line 410, there may be one or more production line checks 420 performed. After the analytical laboratory instrument 404 is produced, it may be inspected and the results stored in a final goods inspection 422. The analytical laboratory instrument 404 may then be released for distribution to a particular customer, which may be reflected in a customer release 424.

After the analytical laboratory instrument 404 is delivered to the laboratory 402, a field service engineer may inspect the analytical laboratory instrument 404 to determine that it is set up properly, and may calibrate (or verify the calibration of) the analytical laboratory instrument 404. The results of these checks may be stored in a field service engineer report 426. The analytical laboratory instrument 404 may be subject to ongoing preventative maintenance and service, the records of which may be stored in an annual service report 428. If there is a problem with the analytical laboratory instrument 404 and the problem is remedied through remote service, the service procedures may be documented in a remote service report 430. If there is a problem that requires a service technician to repair the device, this may be documented in a maintenance incident report 432. Any or all of these documents may be useful for auditing purposes.

Any or all of the instrument design 414, materials manifest 416, materials inspection 418, production line checks 420, final goods inspection 422, customer release 424, field service engineer report 426, annual service report 428, remote service report 430, maintenance incident report 432, instrument standard 436, scientific paper 446, material records 442, method 448 may need to be signed by one or more authorizing users. The signature requirement may represent a quality gate so that, when the document is signed, it triggers a record to be created in the chain of authority.

The record may be based on a predecessor record (unless the record represents a root node, such as the instrument standard 436, scientific paper 446, and optionally the instrument design 414 which might or might not depend from a particular instrument standard 436). For example, an “instrument” node in the tree may represent the analytical laboratory instrument 404, which may be linked back to predecessor nodes describing the raw materials 440, instrument design 414, etc.

The immediate predecessor record (or multiple predecessor records) may be used to secure the current record, such as by computing a cryptographic hash over the predecessor record(s) and storing the hash with the current record. In this way, if the predecessor record(s) are changed the change will be immediately detectable because a hash computed over the updated predecessor record(s) will not match the hash stored in the current record.

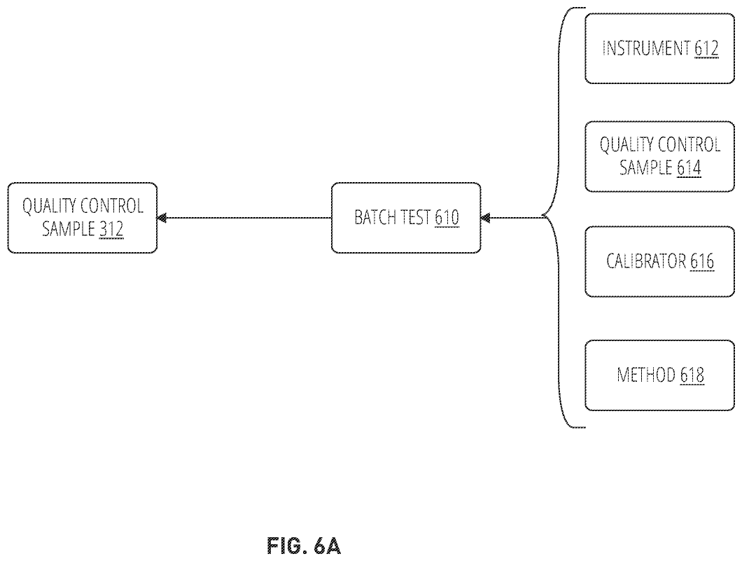

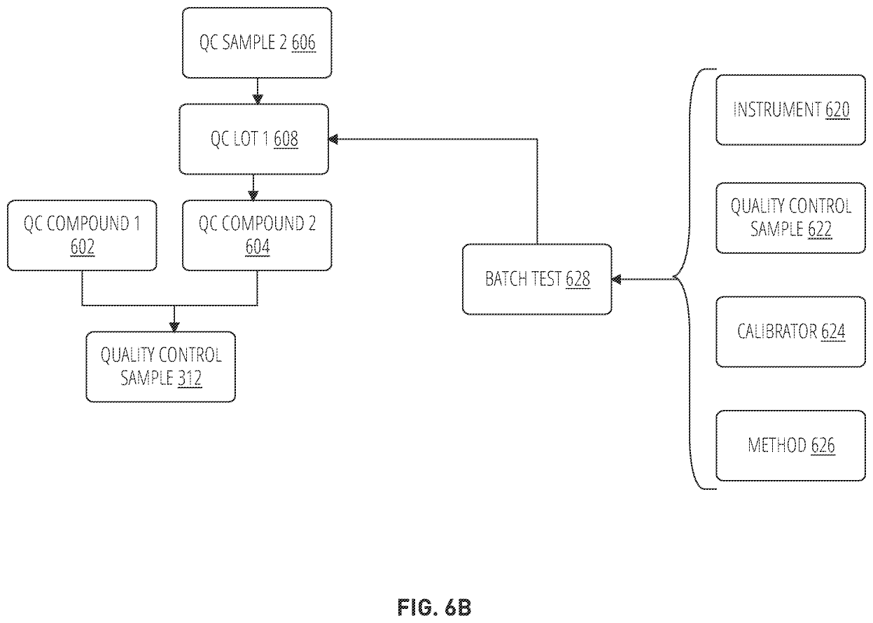

FIG. 4 illustrates how a particular node dedicated to an auditable parameter (e.g., the instrument 306 auditable parameter node from FIG. 3) might link back to particular predecessor nodes. Of course, different instruments analytical laboratory instrument 404 may have different production and use contexts, and so may include more, fewer, or different sets of nodes. Similarly, sets of linked nodes can be generated for the reagents 450 tested by the analytical laboratory instrument 404, the method 448 used to analyze the data, etc. Although these trees are not shown in FIG. 4 due to space constraints, they would be present in the chain of authority in the same way as the records related to the analytical laboratory instrument 404. An example of a tree directed to a method 308 is shown in FIG. 5, while trees directed to various reagents are shown in FIG. 6A and FIG. 6B. The analytical laboratory instrument 404 may be comprised of one or more subassemblies, modules, or optional components, each having information as described above regarding analytical laboratory instrument 404, which together provide a record depicting the configuration of analytical laboratory instrument 404.

FIG. 5 depicts an example of a set of nodes in the form of a tree that represents a chain of authority for a method 308 auditable parameter. The method 308 may represent an analytical method or workflow. The method 308 may have been developed over a period of time. For instance, a user or group of users may propose a first version 508 of the method 308, which was then revised into a second version 510 of the method 308. As each version of the method was marked as complete, an associated record may be created in the chain of authority.

The second version 510 of the method 308 may have been reviewed by a reviewer, who approved the second version 510 and signed off on it. The signature may trigger the creation of a new record representing the approved second version 512 of the method 308.

In order for the reviewer to approve the second version 510 of the method 308 to generate the approved second version 512, a number of tests may have been done. For example, the reviewer may have used the second version 510 of the method 308 to test a known sample 102 in order to validate that the second version 510 of the method 308 produces expected analytical results. The sample 102 may have been tested using an instrument 306; the instrument 306 may have been subjected to suitability tests based on a quality control sample 312 and may have been calibrated using a calibrator 310.

When the sample 102 is tested, it may generate validation results 502. The validation results 502 may be compared to a predetermined specification 504. Whether the validation results 502 sufficiently matches the specification 504 in order to approve the second version 510 may depend on thresholds or acceptable windows as defined by risk management requirements 506.

Each of the first version 508, second version 510, approved second version 512, quality control sample 312, calibrator 310, sample 102, instrument 306, validation results 502, specification 504, and risk management requirements 506 may represent auditable parameters, each associated with a node representing a record in a tree or blockchain. Each may be associated with one or more documents or digital information (e.g., signatures, data, etc.). Furthermore, each of these nodes (especially, for example, the instrument 306, quality control sample 312, calibrator 310, and sample 102) may be associated with their own sub-trees (e.g., the instrument 306 used to test the sample 102 in accordance with the second version 510 of the method 308 might be associated with a tree like the one depicted in FIG. 4; the reagents including the quality control sample 312, calibrator 310, and sample 102 might be associated with a tree like the ones depicted in FIG. 6A or FIG. 6B).

FIG. 6A depicts an example of a set of nodes in the form of a tree that represents a chain of authority for a reagent auditable parameter. In this example, the reagent is a quality control sample 312, which is tested in a batch test 610. The batch test 610 may be performed by an instrument 612 according to a method 618. The instrument 612 may be calibrated by a calibrator 616 and may be validated by a quality control sample 614, which may be the same as or different than the quality control sample 312. Each of the depicted nodes may serve as a record in a tree or blockchain.

The quality control sample 312 may itself be made up of several reagents, and so the node corresponding to the quality control sample 312 may be zoomed-into or drilled down on in order to see the nodes related to those reagents. For instance, FIG. 6B depicts how the tree for the reagent auditable parameter of FIG. 6A can be further broken down or enhanced by zooming into the node corresponding to the quality control sample 312.

As shown, the quality control sample 312 is made up of a QC Compound 1 602 and a QC Compound 2 604. The QC Compound 2 604 may itself include several reagents, such as QC Sample 2 606. A QC Lot 1 608 of the QC Sample 2 606 may have been tested in a batch test 628 using an instrument 620 according to a method 626. The instrument 620 may be calibrated using a calibrator 624 and validated using a quality control sample 622. Each of these nodes may represent records in a tree or blockchain.

The trees of FIG. 6A and FIG. 6B (as well as the other trees depicted and described above) may be displayed in a graphical user interface. Users may select one or more of the nodes to zoom in on that node and display any sub-trees associated with those nodes (and this process may be repeated for any node that is not a root node, such as a standard or academic paper). Any documents, signatures, data, or other information associated with a node may be retrieved for display when a user selects that node—for instance, selecting the specification 504 node of FIG. 5 may cause the data in the specification 504 to be displayed in a suitable interface.

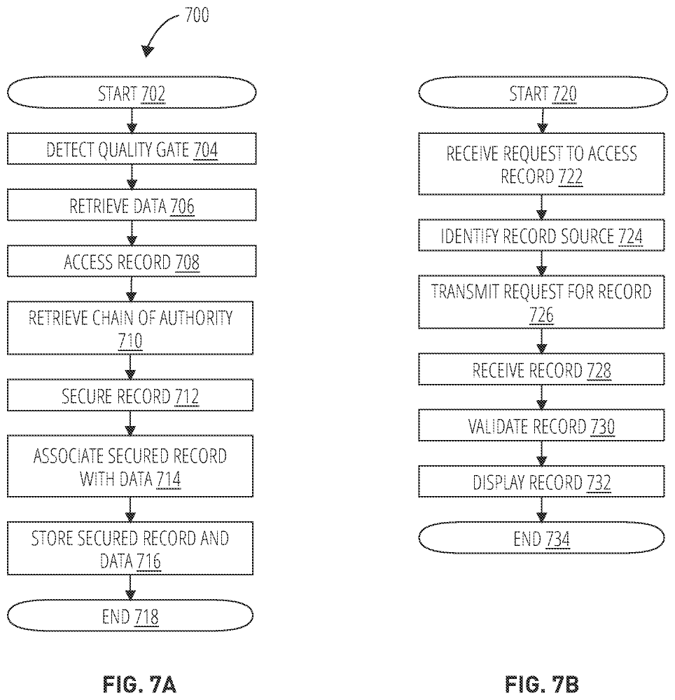

FIG. 7A is a flowchart depicting exemplary node creation logic 700 for creating a node in a tree or blockchain corresponding to a record according to an exemplary embodiment. The logic may be embodied as instructions stored on a computer-readable medium configured to be executed by a processor. The logic may be implemented by a suitable computing system configured to perform the actions described below.

Processing begins at start block 702. For example, processing may start when a user logs into a computing system configured to log information about auditable parameters relating to an analytical laboratory instrument. An auditable parameter may be, for example, a reagent, an instrument, a method, a calibration, or a quality control action.

At start block 702, a process may begin operating in the background of the system. This background process may set up a listener that is subscribed to receive events; such an event may be triggered when a quality gate (as described above) is reached. For example, a quality gate may be reached when a signature is received relating to an auditable parameter, when a document or data is generated for an auditable parameter, etc. At block 704, the quality gate may be detected.

At block 706, the system may retrieve data associated with the quality gate. For example, if the quality gate was triggered by a user signing a document or a method, a copy of the document or a representation of the method may be retrieved (potentially along with the signature). Similarly, if the quality gate was triggered by the collection of data, the data may be retrieved.

At block 708, the system may access or create a record associated with the quality gate. For example, if the quality gate was triggered by the approval of a new method, a new record for the method may be created and accessed. If the quality gate was triggered by the collection of data by an analytical laboratory instrument, a record associated with the instrument may be accessed. If a reagent was tested as part of the data collection, a record associated with the reagent may be created or accessed.

The records may represent nodes in a tree, such as a Merkle tree or blockchain, in which the nodes are secured by a cryptographic hash based on respective content of one or more preceding nodes.

The record may be configured so as to be compliant with an audit requirement defined by a regulatory or scientific authority. For example, an authority may require that certain data be preserved at specified times, that signatures be provided and recorded upon the occurrence of certain events, or that an explanation be collected when certain actions are taken. The particular auditing requirements will vary with the field of use and the regulatory authority (for instance, the US Food and Drug Administration may have different auditing requirements than The Medicines and Healthcare products Regulatory Agency MHRA of the United Kingdom UK).

The record may include a chain of authority tracing the auditable parameter that triggered the quality gate in block 704 backwards to a previous auditable parameter, which may be retrieved in block 710. For example, the new method may have been based on a previous method and may have been tested against a specification (as depicted in FIG. 5). Records for the previous method and the specification (as well as related auditable parameters such as the instrument used to test a sample against the specification) may have been created when a quality gate associated with these auditable parameters was previously reached. Each of these records may represent previous auditable parameters that are linked to the current record in the chain of authority. The chain of authority may be identified and configured automatically (e.g., by automatically detecting an identity of the instrument used to test the method) or manually (e.g., by presenting an interface allowing a user to connect records to each other in a tree structure). In some embodiments, the system may attempt to build a tree where each node connects back to a root representing a standard, a pharmacopeia, a scientific paper, a regulation, or design data for a reagent or a part of the laboratory analytical instrument.

The records of the previous auditable parameters may include data or other information. At block 712, the system may secure the current record based on the previous records so that a change in the previous records can be detected. For example, a hash value may be computed over the data or other information of the previous records, and this hash value may be stored with the record. The record may include an identifier or link to any previous records connected to the current record.

The record may or may not be embedded with the data or information retrieved at block 706. If the record is embedded with this data or information, they may be stored together in a same data structure. Thus, retrieving the data or information may cause the record to also be retrieved. In some embodiments, the data or information may be associated with the record without being embedded; for example, the record may include a pointer to the location where the data or information can be located.

At block 716, the secured record and data may be stored in a non-transitory computer readable medium. Processing may then proceed to block 718 and end.

FIG. 7B is a flowchart depicting exemplary logic for retrieving and displaying records in a tree or blockchain according to an exemplary embodiment. The logic may be embodied as instructions stored on a computer-readable medium configured to be executed by a processor. The logic may be implemented by a suitable computing system configured to perform the actions described below.

At block 722, the system may receive a request to access a record. For example, the system may display a graphical user interface configured to display the above-described tree in a graphical form. If a user selects one of the nodes in the tree, the system may register this as a request to access the record associated with the node.

At block 724, the system may identify a source of the record. The source of the record may be an entity that created the record, or a different entity responsible for maintaining the data or information associated with the record. The source of the record may be identified in the record.

If the source of the record is the same as the entity that accessed the record, then the data or information associated with the record may be directly available to the entity and the requesting entity may proceed to retrieve the record. Otherwise, the entity may need to submit a request for the record (block 726), which may include authorization information (e.g., login credentials) confirming the access rights of the entity with respect to the record. Assuming that the owner of the record authorizes the access, then at block 728, the system may receive a copy of the record in response to the request.

The received record may include a hash value calculated over the data of previous records. Optionally, at block 730 the system may confirm that the record is valid by hashing the data or information of the previous records and comparing the resulting hash value to the hash value retrieved from the record. If the two do not match, then it is known that the previous data (or the hash value) has been altered since the record was created. In a similar manner, the data or information associated with the retrieved record can be validated by retrieving a subsequent record (a record depending from the requested record) and comparing the hash value in the subsequent record (which was computed based on the retrieved record) with a hash value calculated over the retrieved record.

Assuming that the data or information in the retrieved record is validated in block 730, the system may proceed to display the record in the interface in block 732. This may involve displaying a representation of a node corresponding to the auditable parameter and/or the data or information associated with the record.

Block 724 through block 732 may be repeated for any records associated with the requested record. For instance, if a user requests a record related to a method, any or all of the records related to the creation and approval of the method (see, e.g., FIG. 5) may also be retrieved. Similarly, a user may select one of the records displayed in block 732 to zoom in on that record, causing related records (e.g., records connected to the current record in the tree structure) to be retrieved.

FIG. 8 illustrates one example of a system architecture and data processing device that may be used to implement one or more illustrative aspects described herein in a standalone and/or networked environment. Various network nodes, such as the data server 810, web server 806, computer 804, and laptop 802 may be interconnected via a wide area network 808 (WAN), such as the internet. Other networks may also or alternatively be used, including private intranets, corporate networks, LANs, metropolitan area networks (MANs) wireless networks, personal networks (PANs), and the like. Network 808 is for illustration purposes and may be replaced with fewer or additional computer networks. A local area network (LAN) may have one or more of any known LAN topology and may use one or more of a variety of different protocols, such as ethernet. Devices data server 810, web server 806, computer 804, laptop 802 and other devices (not shown) may be connected to one or more of the networks via twisted pair wires, coaxial cable, fiber optics, radio waves or other communication media.

Computer software, hardware, and networks may be utilized in a variety of different system environments, including standalone, networked, remote-access (aka, remote desktop), virtualized, and/or cloud-based environments, among others.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data-attributable to a single entity-which resides across all physical networks.

The components may include data server 810, web server 806, and client computer 804, laptop 802. Data server 810 provides overall access, control and administration of databases and control software for performing one or more illustrative aspects described herein. Data server 810 may be connected to web server 806 through which users interact with and obtain data as requested. Alternatively, data server 810 may act as a web server itself and be directly connected to the internet. Data server 810 may be connected to web server 806 through the network 808 (e.g., the internet), via direct or indirect connection, or via some other network. Users may interact with the data server 810 using remote computer 804, laptop 802, e.g., using a web browser to connect to the data server 810 via one or more externally exposed web sites hosted by web server 806. Client computer 804, laptop 802 may be used in concert with data server 810 to access data stored therein or may be used for other purposes. For example, from client computer 804, a user may access web server 806 using an internet browser, as is known in the art, or by executing a software application that communicates with web server 806 and/or data server 810 over a computer network (such as the internet).

Servers and applications may be combined on the same physical machines, and retain separate virtual or logical addresses, or may reside on separate physical machines. FIG. 8 illustrates just one example of a network architecture that may be used, and those of skill in the art will appreciate that the specific network architecture and data processing devices used may vary, and are secondary to the functionality that they provide, as further described herein. For example, services provided by web server 806 and data server 810 may be combined on a single server.

Each component data server 810, web server 806, computer 804, laptop 802 may be any type of known computer, server, or data processing device. Data server 810, e.g., may include a processor 812 controlling overall operation of the data server 810. Data server 810 may further include RAM 816, ROM 818, network interface 814, input/output interfaces 820 (e.g., keyboard, mouse, display, printer, etc.), and memory 822. Input/output interfaces 820 may include a variety of interface units and drives for reading, writing, displaying, and/or printing data or files. Memory 822 may further store operating system software 824 for controlling overall operation of the data server 810, control logic 826 for instructing data server 810 to perform aspects described herein, and other application software 828 providing secondary, support, and/or other functionality which may or may not be used in conjunction with aspects described herein. The control logic may also be referred to herein as the data server software control logic 826. Functionality of the data server software may refer to operations or decisions made automatically based on rules coded into the control logic, made manually by a user providing input into the system, and/or a combination of automatic processing based on user input (e.g., queries, data updates, etc.).

Memory 1122 may also store data used in performance of one or more aspects described herein, including a first database 832 and a second database 830. In some embodiments, the first database may include the second database (e.g., as a separate table, report, etc.). That is, the information can be stored in a single database, or separated into different logical, virtual, or physical databases, depending on system design. Web server 806, computer 804, laptop 802 may have similar or different architecture as described with respect to data server 810. Those of skill in the art will appreciate that the functionality of data server 810 (or web server 806, computer 804, laptop 802) as described herein may be spread across multiple data processing devices, for example, to distribute processing load across multiple computers, to segregate transactions based on geographic location, user access level, quality of service (QoS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution or may be written in a scripting language such as (but not limited to) HTML or XML. The computer executable instructions may be stored on a computer readable medium such as a nonvolatile storage device. Any suitable computer readable storage media may be utilized, including hard disks, CD-ROMs, optical storage devices, magnetic storage devices, and/or any combination thereof. In addition, various transmission (non-storage) media representing data or events as described herein may be transferred between a source and a destination in the form of electromagnetic waves traveling through signal-conducting media such as metal wires, optical fibers, and/or wireless transmission media (e.g., air and/or space). various aspects described herein may be embodied as a method, a data processing system, or a computer program product. Therefore, various functionalities may be embodied in whole or in part in software, firmware and/or hardware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general-purpose computer as selectively activated or reconfigured by a computer program stored in the computer. The procedures presented herein are not inherently related to a particular computer or other apparatus. Various general-purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general-purpose computer as selectively activated or reconfigured by a computer program stored in the computer. The procedures presented herein are not inherently related to a particular computer or other apparatus. Various general-purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A computer implemented method, comprising:
receiving analytical data from a laboratory analytical instrument, wherein the analytical data is generated based on an auditable parameter;
accessing a record for the auditable parameter, wherein the record includes a chain of authority tracing the auditable parameter backwards to a previous auditable parameter;
cryptographically securing the record based on the chain of authority to generate a cryptographically secured record; and
associating the cryptographically secured record with the data.

2. The computer-implemented method of claim 1, wherein the auditable parameter comprises an identification of at least one of a reagent, an instrument, a method, a calibration, or a quality control action.

3. The computer-implemented method of claim 1, wherein the record is compliant with an audit requirement defined by a regulatory or scientific authority.

4. The computer-implemented method of claim 1, wherein the record traces back to a root comprising a standard, a pharmacopeia, a scientific paper, a regulation, or design data for a reagent or a part of the laboratory analytical instrument.

5. The computer-implemented method of claim 1, wherein the auditable parameter and previous auditable parameter are each associated with a quality gate that triggers a respective record to be created.

6. The computer-implemented method of claim 1, wherein the record is in the form of a tree having nodes secured by a cryptographic hash based on respective content of one or more preceding nodes.

7. The computer-implemented method of claim 1, wherein at least one record along the chain of authority is maintained by a third party, and further comprising:
receiving a request to access the at least one record;
transmitting the request to the third party;
receiving the at least one record in response to the request, the at least one record secured by a cryptographic hash; and
validating the at least one record using the cryptographic hash.

8. A non-transitory computer-readable storage medium, the computer-readable storage medium including instructions that when executed by a computer, cause the computer to:
receive analytical data from a laboratory analytical instrument, wherein the analytical data is generated based on an auditable parameter;
access a record for the auditable parameter, wherein the record includes a chain of authority tracing the auditable parameter backwards to a previous auditable parameter;
cryptographically secure the record based on the chain of authority to generate a cryptographically secured record; and
associate the cryptographically secured record with the data.

9. The computer-readable storage medium of claim 8, wherein the auditable parameter comprises an identification of at least one of a reagent, an instrument, a method, a calibration, or a quality control action.

10. The computer-readable storage medium of claim 8, wherein the record is compliant with an audit requirement defined by a regulatory or scientific authority.

11. The computer-readable storage medium of claim 8, wherein the record traces back to a root comprising a standard, a pharmacopeia, a scientific paper, a regulation, or design data for a reagent or a part of the laboratory analytical instrument.

12. The computer-readable storage medium of claim 8, wherein the auditable parameter and previous auditable parameter are each associated with a quality gate that triggers a respective record to be created.

13. The computer-readable storage medium of claim 8, wherein the record is in the form of a tree having nodes secured by a cryptographic hash based on respective content of one or more preceding nodes.

14. The computer-readable storage medium of claim 8, wherein at least one record along the chain of authority is maintained by a third party, and wherein the instructions further configure the computer to:
receive a request to access the at least one record;
transmit the request to the third party;
receive the at least one record in response to the request, the at least one record secured by a cryptographic hash; and
validate the at least one record using the cryptographic hash.

15. A computing apparatus comprising:
a processor; and
a memory storing instructions that, when executed by the processor, configure the apparatus to:
receive analytical data from a laboratory analytical instrument, wherein the analytical data is generated based on an auditable parameter;
access a record for the auditable parameter, wherein the record includes a chain of authority tracing the auditable parameter backwards to a previous auditable parameter;
cryptographically secure the record based on the chain of authority to generate a cryptographically secured record; and
associate the cryptographically secured record with the data.

16. The computing apparatus of claim 15, wherein the auditable parameter comprises an identification of at least one of a reagent, an instrument, a method, a calibration, or a quality control action.

17. The computing apparatus of claim 15, wherein the record is compliant with an audit requirement defined by a regulatory or scientific authority.

18. The computing apparatus of claim 15, wherein the record traces back to a root comprising a standard, a pharmacopeia, a scientific paper, a regulation, or design data for a reagent or a part of the laboratory analytical instrument.

19. The computing apparatus of claim 15, wherein the auditable parameter and previous auditable parameter are each associated with a quality gate that triggers a respective record to be created.

20. The computing apparatus of claim 15, wherein at least one record along the chain of authority is maintained by a third party, and wherein the instructions further configure the apparatus to:
receive a request to access the at least one record;
transmit the request to the third party;
receive the at least one record in response to the request, the at least one record secured by a cryptographic hash; and
validate the at least one record using the cryptographic hash.
